(19) United States
(12) Patent Application Publication
(10) Pub. No.: US 2023/0003708 A1
(43) Pub. Date: Jan. 5, 2023

(54) METHODS OF GEOLOGIC SAMPLE ANALYSIS

(71) Applicant: Saudi Arabian Oil Company, Dhahran (SA)

(72) Inventors: Bradley Clark Wallet, Norman, OK (US); Tiffany Dawn McAlpin, Richmond, TX (US)

(73) Assignee: Saudi Arabian Oil Company, Dhahran (SA)

(21) Appl. No.: 17/365,533

(22) Filed: Jul. 1, 2021

(51) Int. Cl. G01N 33/24; G01N 1/34; G06T 7/00; E21B 49/08

(52) U.S. Cl. CPC G01N 33/24; G01N 1/34; G06T 7/0004; E21B 49/08; G06T 2207/10024; G06T 2207/30181

(57) ABSTRACT

A method for analyzing a geologic sample includes illuminating the geologic sample with a halogen light beam, and capturing an image of the geologic sample on a camera thereby collecting spectra reflected from a surface of the geologic sample. The method further includes processing the image to identify rock properties of the geologic sample and determine a qualitative descriptor of the rock properties. The method further includes analyzing the qualitative descriptor of the rock properties of the geologic sample to determine subsurface lithography.

### TECHNICAL FIELD

The present specification generally relates to systems and methods for analyzing a geologic sample.

### BACKGROUND

Extracting subsurface fuel sources may require drilling a hole from the surface to the subsurface geological formation housing the fuel. Specialized drilling techniques and materials are utilized to form a wellbore and extract the fuels. A wellbore is a hole that extends from the surface to a location below the surface to permit access to hydrocarbon-bearing subsurface formations. During drilling, samples, known as cuttings, are collected at the rig site and used for preliminary evaluation of reservoir rock quality and hydrocarbon presence. Well site geologists may manually assess hundreds to thousands of cuttings samples per well in order to generate qualitative descriptions and drilling reports. The cuttings provide important constraints for correlation to surrounding stratigraphy, static reservoir models, calculation of hydrocarbons in place and geo-steering decisions. This process is time-consuming which limits the time from cuttings collection to actionable information. In addition, materials characterization may vary from one well-site geologist to the next.

### SUMMARY

Accordingly, a need exists for methods of analyzing geologic samples in realtime to determine rock properties of a geologic sample. The present disclosure addresses this need by incorporating onsite analysis equipment to determine rock properties of a geologic sample. The rock properties may include a color of the geologic sample, a texture of the geologic sample, a shape of the geologic sample, grain size of the geologic sample, or combinations thereof. Onsite analysis equipment allows wellsite geologists to acquire qualitative data quickly and efficiently, reducing the time needed for characterization and analysis at the wellsite. The qualitative data acquired is also consistent over time and at each wellsite with the systems and methods described in this disclosure, therefore making it ideal for qualitative assessment for cuttings analysis.

In accordance with one embodiment of the present disclosure, a method of analyzing a geologic sample includes illuminating the geologic sample with a halogen light beam and capturing an image of the geologic sample on a camera thereby collecting spectra reflected from a surface of the geologic sample. The method further includes processing the image to identify rock properties of the geologic sample and determine a qualitative descriptor of the rock properties of the geologic sample. The method further includes analyzing the qualitative descriptor of the rock properties of the geologic sample to determine subsurface lithography.

### BRIEF DESCRIPTION OF THE DRAWING

The following detailed description of specific embodiments of the present disclosure can be best understood when read in conjunction with the following drawing, in which:

FIG. 1 schematically depicts a system for capturing images according to one or more embodiments described herein.

### DETAILED DESCRIPTION

Embodiments of the present disclosure are directed to methods of analyzing a geologic sample.

A formation is the fundamental unit of lithostratigraphy. As used in the present disclosure, the term “formation” refers to a body of rock that is sufficiently distinctive and continuous from the surrounding rock bodies that the body of rock can be mapped as a distinct entity. A formation is, therefore, sufficiently homogenous to form a single identifiable unit containing similar rheological properties throughout the formation, including, but not limited to, porosity and permeability. A single formation may include different regions, where some regions include hydrocarbons and others do not. To produce hydrocarbons from the hydrocarbon regions of the formation, production wells are drilled to a depth that enables these hydrocarbons to travel from the subsurface formation to the surface.

As stated previously, embodiments of the present disclosure are directed to methods of analyzing a geologic sample. The method may include drilling a wellbore. The method may include preparing a drilling fluid by combining a liquid carrier with a clay-based material. As used throughout this disclosure, the term “clay-based material” can refer to barite, bentonite, barite, or barium sulfate, as nonlimiting examples. These clay-based materials will increase the density or viscosity of the drilling fluid. The drilling fluid may be introduced into the subsurface formation through a drilling assembly. The drilling assembly may include a drilling platform that supports a derrick having a traveling block for raising and lowering a drill string. The drill string may include, but is not limited to, drill pipe and coiled tubing, as generally known to those skilled in the art. As used throughout this disclosure, the term “drill string” refers to the combination of the drill pipe, the bottomhole assembly and any other tools used to drill the wellbore or introduce fluid into the wellbore. As used throughout this disclosure, the term “coiled tubing” refers to a long, continuous length of pipe wound on a spool. The pipe is straightened prior to pushing into a wellbore and rewound to coil the pipe back onto the transport and storage spool. Depending on the pipe base diameter (1 in. to 4½ in.) and the spool size, coiled tubing can range from 2,000 ft to 15,000 ft (610 to 4,570 m) or greater length. A kelly may support the drill string as it is lowered through a rotary table. As used throughout this disclosure, the term “kelly” refers to a long square or hexagonal steel bar with a hole drilled through the middle for a fluid path. The kelly is used to transmit rotary motion from a rotary table or kelly bushing to the drillstring, while allowing the drillstring to be lowered or raised during rotation. A drill bit is attached to the distal end of the drill string and is driven either by a downhole motor or via rotation of the drill string from the well surface, or by both. As the bit rotates, it creates a wellbore that penetrates various subsurface formations.

A pump (e.g., a mud pump) circulates drilling fluid through a feed pipe and to the kelly, which conveys the drilling fluid downhole through the interior of the drill string and through one or more orifices in the drill bit. The drilling fluid is then circulated back to the surface via an annulus defined between the drill string and the walls of the wellbore. As used throughout this disclosure, the term “drill pipe” refers to a tubular steel conduit fitted with special threaded ends called tool joints. The drill pipe connects the rig surface equipment with the bottomhole assembly and the bit, both to pump drilling fluid to the bit and to be able to raise, lower and rotate the bottomhole assembly and bit. At the surface, the recirculated or spent drilling fluid exits the annulus and may be conveyed to one or more fluid processing units via an interconnecting flow line. After passing through the fluid processing units, a “cleaned” drilling fluid is deposited into a nearby mud pit. While illustrated as being arranged at the outlet of the wellbore via the annulus, those skilled in the art will readily appreciate that the fluid processing units may be arranged at any other location in the drilling assembly to facilitate its proper function, without departing from the scope of the disclosure.

The method may include acquiring the geologic sample from the wellbore. In embodiments, the method may further include cleaning the geologic sample. Cleaning the geologic sample may include flushing the geologic sample with a solvent. The solvent may be chosen from the group consisting of acetone, chloroform, methanol, cyclohexane, ethylene chloride, methylene chloride, naphtha, tetrachloroethylene, tetrahydrofuran, toluene, trichloroethylene, xylene, or combinations thereof.

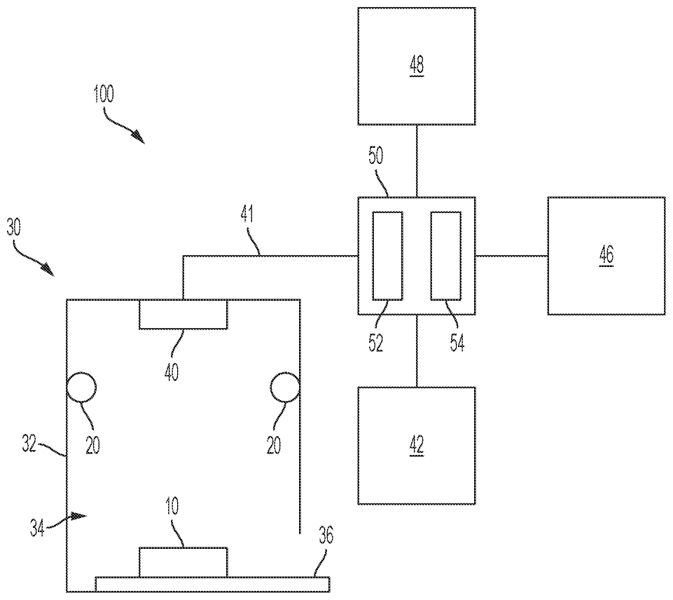

Referring now to FIG. 1, the geologic sample 10 may be placed within a housing 30 of a geologic analysis apparatus 100, where the housing 30 comprises a sidewall 32 at least partially enclosing an imaging chamber 34. In embodiments, placing the geologic sample 10 within the housing 30 includes placing the geologic sample 10 onto an imaging platform 36 positioned within the imaging chamber 34. The imaging platform 36 may be slidably adjustable within the imaging chamber 34 in a horizontal direction.

The geologic sample 10 may then be illuminated with a light beam. The light beam may include a halogen light beam, a polarized light beam, a collimated light beam, or combinations thereof. The light beam may be formed by one or more light sources 20. The one or more light sources 20 may be positioned within the imaging chamber 34 on an interior of the sidewall 32. In embodiments, there may be two light sources 20, as shown in FIG. 1. In embodiments, there may be one light source 20 (not shown) or more than two light sources 20 (not shown), such as three, four, five, six, or seven light sources 20.

As stated previously, the camera 40 may include an image sensor and a focusing lens. In some embodiments, the focusing lens may be a telecentric lens. Conventional lenses have angular fields of view, which means that as the distance between the lens and object increase, the magnification decreases. This angular field of view results in parallax error, also known as perspective error. Telecentric lenses eliminate this parallax error by having a constant, non-angular field of view. With telecentric lenses, magnification remains constant with object displacement, provided the object stays within the telecentric range, meaning the total distance above and below an object that remains in focus and at constant magnification.

The focusing lens may be optically coupled to the image sensor. The image sensor includes a hyperspectral camera. The image sensor may have a pixel count of at least 10 megapixels, allowing the image sensor to capture high resolution images. In embodiments, the pixel count may be as low as 16 by 16 pixels, 256 by 256 pixels, 640 by 480 pixels, 1024 by 768 pixels, 1280 by 1024 pixels, 1920 by 1080 pixels, or 4064 by 2704 pixels. In embodiments, the image sense may have a pixel count of from 1 to 100 megapixels, from 1 to 75 megapixels, from 1 to 50 megapixels, from 1 to 25 megapixels, from 1 to 10 megapixels, from 10 to 100 megapixels, from 10 to 75 megapixels, from 10 to 50 megapixels, from 10 to 25 megapixels, from 25 to 100 megapixels, from 25 to 75 megapixels, from 25 to 50 megapixels, from 50 to 100 megapixels, from 50 to 75 megapixels, or from 75 to 100 megapixels.

In embodiments, the halogen light beam 20 and the camera 40 may be positioned on or above the geologic sample 10, as shown in FIG. 1. In embodiments, the light source 20 and the camera 40 may be positioned on opposite sides of the geologic sample 10, such that the halogen light beam may pass through the geologic sample 10 to be captured by the focusing lens and the image sensor.

In embodiments, the camera 40 may acquire images of the geologic sample and the method further includes capturing an image of the geologic sample 10 on the camera 40, thereby collecting spectra reflected from a surface of the geologic sample 10. The images acquired by the camera 40 may be processed to identify rock properties of the geologic sample and determine a qualitative descriptor of the rock properties.

The wavelengths of the spectra reflected from the surface of the geologic sample 10 may range from 100 to 15000 nanometers (nm), from 100 to 14000 nm, from 100 to 13000 nm, from 100 to 12000 nm, from 100 to 11000 nm, from 100 to 10000 nm, from 100 to 9000 nm, from 100 to 8000 nm, from 100 to 7000 nm, from 100 to 6000 nm, from 100 to 5000 nm, from 100 to 4000 nm, from 100 to 3000 nm, from 100 to 2000 nm, from 100 to 1000 nm, from 100 to 800 nm, from 100 to 600 nm, from 100 to 400 nm, from 100 to 250 nm, from 250 to 15000 nm, from 250 to 14000 nm, from 250 to 13000 nm, from 250 to 12000 nm, from 250 to 11000 nm, from 250 to 10000 nm, from 250 to 9000 nm, from 250 to 8000 nm, from 250 to 7000 nm, from 250 to 6000 nm, from 250 to 5000 nm, from 250 to 4000 nm, from 250 to 3000 nm, from 250 to 2000 nm, from 250 to 1000 nm, from 250 to 800 nm, from 250 to 600 nm, from 250 to 400 nm, from 400 to 15000 nm, from 400 to 14000 nm, from 400 to 13000 nm, from 400 to 12000 nm, from 400 to 11000 nm, from 400 to 10000 nm, from 400 to 9000 nm, from 400 to 8000 nm, from 400 to 7000 nm, from 400 to 6000 nm, from 400 to 5000 nm, from 400 to 4000 nm, from 400 to 3000 nm, from 400 to 2000 nm, from 400 to 1000 nm, from 400 to 800 nm, from 400 to 600 nm, from 600 to 15000 nm, from 600 to 14000 nm, from 600 to 13000 nm, from 600 to 12000 nm, from 600 to 11000 nm, from 600 to 10000 nm, from 600 to 9000 nm, from 600 to 8000 nm, from 600 to 7000 nm, from 600 to 6000 nm, from 600 to 5000 nm, from 600 to 4000 nm, from 600 to 3000 nm, from 600 to 2000 nm, from 600 to 1000 nm, from 600 to 800 nm, from 800 to 15000 nm, from 800 to 14000 nm, from 800 to 13000 nm, from 800 to 12000 nm, from 800 to 11000 nm, from 800 to 10000 nm, from 800 to 9000 nm, from 800 to 8000 nm, from 800 to 7000 nm, from 800 to 6000 nm, from 800 to 5000 nm, from 800 to 4000 nm, from 800 to 3000 nm, from 800 to 2000 nm, from 800 to 1000 nm, from 1000 to 15000 nm, from 1000 to 14000 nm, from 1000 to 13000 nm, from 1000 to 12000 nm, from 1000 to 11000 nm, from 1000 to 10000 nm, from 1000 to 9000 nm, from 1000 to 8000 nm, from 1000 to 7000 nm, from 1000 to 6000 nm, from 1000 to 5000 nm, from 1000 to 4000 nm, from 1000 to 3000 nm, from 1000 to 2000 nm, from 2000 to 15000 nm, from 2000 to 14000 nm, from 2000 to 13000 nm, from 2000 to 12000 nm, from 2000 to 11000 nm, from 2000 to 10000 nm, from 2000 to 9000 nm, from 2000 to 8000 nm, from 2000 to 7000 nm, from 2000 to 6000 nm, from 2000 to 5000 nm, from 2000 to 4000 nm, from 2000 to 3000 nm, from 3000 to 15000 nm, from 3000 to 14000 nm, from 3000 to 13000 nm, from 3000 to 12000 nm, from 3000 to 11000 nm, from 3000 to 10000 nm, from 3000 to 9000 nm, from 3000 to 8000 nm, from 3000 to 7000 nm, from 3000 to 6000 nm, from 3000 to 5000 nm, from 3000 to 4000 nm, from 4000 to 15000 nm, from 4000 to 14000 nm, from 4000 to 13000 nm, from 4000 to 12000 nm, from 4000 to 11000 nm, from 4000 to 10000 nm, from 4000 to 9000 nm, from 4000 to 8000 nm, from 4000 to 7000 nm, from 4000 to 6000 nm, from 4000 to 5000 nm, from 5000 to 15000 nm, from 5000 to 14000 nm, from 5000 to 13000 nm, from 5000 to 12000 nm, from 5000 to 11000 nm, from 5000 to 10000 nm, from 5000 to 9000 nm, from 5000 to 8000 nm, from 5000 to 7000 nm, from 5000 to 6000 nm, from 6000 to 15000 nm, from 6000 to 14000 nm, from 6000 to 13000 nm, from 6000 to 12000 nm, from 6000 to 11000 nm, from 6000 to 10000 nm, from 6000 to 9000 nm, from 6000 to 8000 nm, from 6000 to 7000 nm, from 7000 to 15000 nm, from 7000 to 14000 nm, from 7000 to 13000 nm, from 7000 to 12000 nm, from 7000 to 11000 nm, from 7000 to 10000 nm, from 7000 to 9000 nm, from 7000 to 8000 nm, from 8000 to 15000 nm, from 8000 to 14000 nm, from 8000 to 13000 nm, from 8000 to 12000 nm, from 8000 to 11000 nm, from 8000 to 10000 nm, from 8000 to 9000 nm, from 9000 to 15000 nm, from 9000 to 14000 nm, from 9000 to 13000 nm, from 9000 to 12000 nm, from 9000 to 11000 nm, from 9000 to 10000 nm, from 10000 to 15000 nm, from 10000 to 14000 nm, from 10000 to 13000 nm, from 10000 to 12000 nm, from 10000 to 11000 nm, from 11000 to 15000 nm, from 11000 to 14000 nm, from 11000 to 13000 nm, from 11000 to 12000 nm, from 12000 to 15000 nm, from 12000 to 14000 nm, from 12000 to 13000 nm, from 13000 to 15000 nm, from 13000 to 14000 nm, or from 14000 to 15000 nm.

In embodiments, the geologic sample 10 is positioned within a field of view of the camera 40. The “field of view” as used herein refers to size of the area on a plane perpendicular to the optical path in the object space at a distance along the optical path that is in focus being imaged on the image sensor.

In embodiments, the image of the geologic sample 10 captured on the image sensor may be captured in R-G-B color space. In embodiments, the image may include an image of a surface of the geologic sample 10. For example, the image may include an image of the front surface 12 of the geologic sample 10.

In embodiments, the method may further include processing the image to identify rock properties of the geologic sample 10 and determine a qualitative descriptor of the rock properties. The camera 40 may be communicatively connected via a communication path 41 to a controller 50, an operating device 46, a power supply 48, and a display device 42. In embodiments, the communication path 41 may communicatively couple each of these components to each other.

The controller 50 comprises a processor 52 and a non-transitory electronic memory 54 to which various components are communicatively coupled. In some embodiments, the processor 52 and the non-transitory electronic memory 54 and/or the other components are included within a single device. In other embodiments, the processor 52 and the non-transitory electronic memory 54 and/or the other components may be distributed among multiple devices that are communicatively coupled. The controller 50 includes non-transitory electronic memory 54 that stores a set of machine-readable instructions. The processor 52 executes the machine-readable instructions stored in the non-transitory electronic memory 54. The non-transitory electronic memory 54 may comprise RAM, ROM, flash memories, hard drives, or any device capable of storing machine-readable instructions such that the machine-readable instructions can be accessed by the processor 52. Accordingly, the controller 50 described herein may be implemented in any conventional computer programming language, as pre-programmed hardware elements, or as a combination of hardware and software components. The non-transitory electronic memory 54 may be implemented as one memory module or a plurality of memory modules.

In some embodiments, the non-transitory electronic memory 54 includes instructions for executing the functions of processing the image to identify rock properties of the geologic sample 10 and determining a qualitative descriptor of the rock properties.

The processor 52 may be any device capable of executing machine-readable instructions. For example, the processor 52 may be an integrated circuit, a microchip, a computer, or any other computing device. The non-transitory electronic memory 54 and the processor 52 are coupled to the communication path 41 that provides signal interconnectivity between each component. Accordingly, the communication path 41 may communicatively couple any number of processors with one another, and allow the modules coupled to the communication path 41 to operate in a distributed computing environment. Specifically, each of the modules may operate as a node that may send and/or receive data. As used herein, the term “communicatively coupled” means that coupled components are capable of exchanging data signals with one another such as, for example, electrical signals via conductive medium, electromagnetic signals via air, optical signals via optical waveguides, and the like.

The communication path 41 communicatively couples the processor 52 and the non-transitory electronic memory 54 of the controller 50 with a plurality of other components, such as the operating device 46. The operating device 46 allows for a user to control operation of the geologic analysis apparatus 100. In some embodiments, the operating device 46 may be a switch, toggle, button, or any combination of controls to provide user operation. The operating device 46 is coupled to the communication path 41 such that the communication path 41 communicatively couples the operating device 46 to other modules. The operating device 46 may provide a user interface for receiving user instructions as to a specific operating of the geologic analysis apparatus 100, such as processing the image to identify specific rock properties of the geologic sample 10, such as color, texture, shape, grain size, or combinations thereof. The operating device 46 may further provide a user interface for determining a qualitative descriptor of the identified rock properties.

The communication path 41 communicatively couples the processor 52 and the non-transitory electronic memory 54 of the controller 50 with a plurality of other components, such as the power supply 48. The power supply 48 (e.g., battery) provides power to the geologic analysis apparatus 100. In some embodiments, the power supply 48 is a rechargeable direct current power source. It is to be understood that the power supply 48 may be a single power supply or battery for providing power to the geologic analysis apparatus 100. A power adapter (not shown) may be provided and electrically coupled via a wiring harness or the like for providing power to the geologic analysis apparatus 100 via the power supply 48.

In some embodiments, the geologic analysis apparatus 100 also includes a display device 42. The display device 42 is coupled to the communication path 41 such that the communication path 41 communicatively couples the display device 42 to other modules of the geologic analysis apparatus 100. Accordingly, the display device 42 may include the operating device 46 and receive mechanical input directly upon the optical output provided by the display device 42. In embodiments, the display device 42 may display the qualitative descriptor determined from processing the image. The qualitative descriptor may describe the color, texture, shape, grain size, or combinations thereof of the geologic sample 10. The qualitative descriptor in regards to color may include red, orange, yellow, green, cyan, blue, indigo, violet, pink, brown, white, gray, black, or combinations thereof. The qualitative descriptor in regards to texture may include rough, smooth, pebbled, glassy, porphyritic, course-grained, fine-grained, or combinations thereof. The qualitative descriptor in regards to shape may include spherical, round, square, angular, or combinations thereof. The qualitative descriptor in regards to grain size may include small, medium, large, or combinations thereof.

The method further includes analyzing the qualitative descriptor of the rock properties of the geologic sample 10 to determine subsurface lithography. Mud logging is an important step in the real time monitoring of drilling a well. By analyzing these properties, it is possible to determine the specific subsurface lithology and fluids being encountered as the well is being drilled. Determining the subsurface lithology and/or the fluids present within that subsurface lithology as the well is being drilled may allow the specific lithography/reservoir characteristics of the subsurface formation to be determined in real time as that depth is being encountered, allowing decisions to be made. For example, and not by way of limitation, those decisions may include adjusting drilling operations depending on the subsurface lithography. Adjusting drilling operations may include changing drilling parameters depending on where the drillbit is relative to the known or expected geology, deciding whether or not it is productive to continue drilling, and deciding in real time when the well has reached the target depth, the target formation, the target reservoir, or combinations thereof.

### EXAMPLES

### Example 1

A geologic sample was placed within a geologic analysis apparatus as described in this disclosure. The geologic sample was illuminated by an AmScope LED 144B-ZK (available from AmScope headquartered in Irvine, Calif.). An image of the geologic sample was then captured on an ArduCam UC 261 camera (available from ArduCam headquartered in Nanjing, China). The image was then processed by a Raspberry Pi 4 Model B computer (available from The Raspberry Pi Foundation based in the United Kingdom) to identify the color of the geologic sample and determine a qualitative descriptor of the color. The color was identified as 65, 44, 31 on the RGB value scale (ranging from 0 to 255). The qualitative descriptor was “red.”

### Example 2

Another geologic sample was placed within a geologic analysis apparatus as described in this disclosure. The geologic sample was illuminated by an AmScope LED 144B-ZK (available from AmScope headquartered in Irvine, Calif.). An image of the geologic sample was then captured on an ArduCam UC 261 camera (available from ArduCam headquartered in Nanjing, China). The image was then processed by a Raspberry Pi 4 Model B computer (available from The Raspberry Pi Foundation based in the United Kingdom) to identify the color of the geologic sample and determine a qualitative descriptor of the color. The color was identified as 0, 62, 38 on the RGB value scale (ranging from 0 to 255). The qualitative descriptor was “cyan.”

For the purposes of describing and defining the embodiments, it is noted that reference herein to a variable being a “function” of a parameter or another variable is not intended to denote that the variable is exclusively a function of the listed parameter or variable. Rather, reference herein to a variable that is a “function” of a listed parameter is intended to be open ended such that the variable may be a function of a single parameter or a plurality of parameters.

Directional terms as used herein—for example up, down, right, left, front, back, top, bottom—are made only with reference to the figures as drawn and are not intended to imply absolute orientation.

Unless otherwise expressly stated, it is in no way intended that any method set forth herein be construed as requiring that its steps be performed in a specific order, nor that with any apparatus specific orientations be required. Accordingly, where a method claim does not actually recite an order to be followed by its steps, or that any apparatus claim does not actually recite an order or orientation to individual components, or it is not otherwise specifically stated in the claims or description that the steps are to be limited to a specific order, or that a specific order or orientation to components of an apparatus is not recited, it is in no way intended that an order or orientation be inferred, in any respect. This holds for any possible non-express basis for interpretation, including: matters of logic with respect to arrangement of steps, operational flow, order of components, or orientation of components; plain meaning derived from grammatical organization or punctuation, and; the number or type of embodiments described in the specification.

For the purposes of defining the present technology, the transitional phrase “consisting of” may be introduced in the claims as a closed preamble term limiting the scope of the claims to the recited components or steps and any naturally occurring impurities. For the purposes of defining the present technology, the transitional phrase “consisting essentially of” may be introduced in the claims to limit the scope of one or more claims to the recited elements, components, materials, or method steps as well as any non-recited elements, components, materials, or method steps that do not materially affect the novel characteristics of the claimed subject matter. The transitional phrases “consisting of” and “consisting essentially of” may be interpreted to be subsets of the open-ended transitional phrases, such as “comprising” and “including,” such that any use of an open ended phrase to introduce a recitation of a series of elements, components, materials, or steps should be interpreted to also disclose recitation of the series of elements, components, materials, or steps using the closed terms “consisting of” and “consisting essentially of.” For example, the recitation of a composition “comprising” components A, B, and C should be interpreted as also disclosing a composition “consisting of” components A, B, and C as well as a composition “consisting essentially of” components A, B, and C. Any quantitative value expressed in the present application may be considered to include open-ended embodiments consistent with the transitional phrases “comprising” or “including” as well as closed or partially closed embodiments consistent with the transitional phrases “consisting of” and “consisting essentially of.”

As used in the Specification and appended Claims, the singular forms “a”, “an”, and “the” include plural references unless the context clearly indicates otherwise. The verb “comprises” and its conjugated forms should be interpreted as referring to elements, components or steps in a non-exclusive manner. The referenced elements, components or steps may be present, utilized or combined with other elements, components or steps not expressly referenced.

It should be understood that any two quantitative values assigned to a property may constitute a range of that property, and all combinations of ranges formed from all stated quantitative values of a given property are contemplated in this disclosure. As used herein, numerical value ranges include the endpoints unless otherwise expressly stated. Thus, for example, stating that “the wavelength may range from 450 to 550 nm” means that the wavelength may be 450 nm, may be 550 nm, or may be any integer between 450 and 550 nm.

The subject matter of the present disclosure has been described in detail and by reference to specific embodiments. It should be understood that any detailed description of a component or feature of an embodiment does not necessarily imply that the component or feature is essential to the particular embodiment or to any other embodiment.

It should be apparent to those skilled in the art that various modifications and variations may be made to the embodiments described within without departing from the spirit and scope of the claimed subject matter. Thus, it is intended that the specification cover the modifications and variations of the various embodiments described within provided such modifications and variations come within the scope of the appended claims and their equivalents. Unless otherwise stated within the application, all tests, properties, and experiments are conducted at room temperature and atmospheric pressure.

It will be apparent to those skilled in the art that various modifications and variations can be made to the embodiments described herein without departing from the spirit and scope of the claimed subject matter. Thus, it is intended that the specification cover the modifications and variations of the various embodiments described herein provided such modification and variations come within the scope of the appended claims and their equivalents.

1. A method of analyzing a geologic sample comprising:
illuminating the geologic sample with a halogen light beam;
capturing an image of the geologic sample on a camera thereby collecting spectra reflected from a surface of the geologic sample;
processing the image to identify rock properties of the geologic sample and determine a qualitative descriptor of the rock properties; and
analyzing the qualitative descriptor of the rock properties of the geologic sample to determine subsurface lithography.

2. The method of claim 1, wherein the rock properties of the geologic sample comprise a color of the geologic sample, a texture of the geologic sample, a shape of the geologic sample, grain size of the geologic sample, or combinations thereof.

3. The method of claim 1, wherein capturing the image comprises capturing the image in R-G-B color space.

4. The method of claim 1, further comprising placing the geologic sample within a housing comprising a sidewall at least partially enclosing an imaging chamber.

5. The method of claim 4, wherein placing the geologic sample within the housing comprises placing the geologic sample onto an imaging platform positioned within the imaging chamber.

6. The method of claim 5, wherein the imaging platform is slidably adjustable within the imaging chamber in a horizontal direction.

7. The method of claim 1, further comprising cleaning the geologic sample prior to illuminating the geologic sample with the halogen light beam.

8. The method of claim 7, wherein cleaning the geologic sample comprises flushing the geologic sample with a solvent.

9. The method of claim 8, wherein the solvent is chosen from the group consisting of acetone, chloroform, methanol, cyclohexane, ethylene chloride, methylene chloride, naphtha, tetrachloroethylene, tetrahydrofuran, toluene, trichloroethylene, xylene, or combinations thereof.

10. The method of claim 1, wherein the camera comprises an image sensor and a lens, and the geologic sample is positioned within a field of view of the camera.

11. The method of claim 10, wherein the image sensor has a pixel count of from 1 to 100 megapixels.

12. The method of claim 1, wherein the spectra reflected from a surface of the geologic sample has a wavelength ranging from 100 to 15000 nm.

13. The method of claim 1, wherein analyzing the qualitative descriptor of the rock properties of the geologic sample to determine subsurface lithography further comprises adjusting drilling operations depending on the subsurface lithography.
